(19) United States
(12) Patent Application Publication
(10) Pub. No.: US 2025/0322515 A1
(43) Pub. Date: Oct. 16, 2025

(54) ENDOSCOPY SUPPORT APPARATUS, METHOD OF OPERATING ENDOSCOPY SUPPORT APPARATUS, AND STORAGE MEDIUM

(71) Applicant: OLYMPUS MEDICAL SYSTEMS CORP., Tokyo (JP)

(72) Inventors: Yoshiko IKEDA, Tokyo (JP); Marina UEZONO, Tokyo (JP)

(73) Assignee: OLYMPUS MEDICAL SYSTEMS CORP., Tokyo (JP)

(21) Appl. No.: 19/174,383

(22) Filed: Apr. 9, 2025

(30) Foreign Application Priority Data
Apr. 12, 2024 (JP) 2024-064641

(51) Int. Cl. G06T 7/00; G06T 7/60; G06T 7/73; G06V 10/764; G16H 40/63

(52) U.S. Cl. CPC G06T 7/0012; G06T 7/60; G06T 7/74; G06V 10/764; G16H 40/63; G06T 2207/10068; G06T 2207/20081; G06T 2207/30092; G06T 2207/30096; G06T 2207/30101; G06T 2207/30204; G06V 2201/031; G06V 2201/07

(57) ABSTRACT

An endoscopy support apparatus includes a memory and a processor. The processor is configured to access the memory that stores: a learned model learned by a learning data set in which a name of a part, a visual field direction, and an axis direction are annotated to each of a plurality of endoscopic image; and a name of at least one target part and a positional relationship of a plurality of parts, and the processor inputs a picked-up image into the learned model, to thereby infer the name of the part, the visual field direction, and the axis direction, in the picked-up image, and outputs a direction of the target part in the picked-up image, based on the positional relationship of the plurality of parts, and the name of the part, the visual field direction, and the axis direction, in the picked-up image, which have been inferred.

### CROSS REFERENCE TO RELATED APPLICATION

This application claims the benefit of Japanese Application No. 2024-064641 filed in Japan on Apr. 12, 2024, the contents of which are incorporated herein by this reference.

### BACKGROUND

### 1. Technical Field

The present disclosure relates to an endoscopy support apparatus for observing a target part in a stomach, a method of operating the endoscopy support apparatus for observing the target part in the stomach, and a storage medium that stores an endoscopy support program for observing the target part in the stomach.

### 2. Description of the Related Art

In a stomach examination using an endoscope system, it is required to observe and photograph a plurality of examination parts in order to prevent overlooking of a lesion.

Japanese Patent Application Laid-Open Publication No. 2010-51399 discloses an endoscopic image recording apparatus that automatically starts and stops recording of endoscopic images. The endoscopic image recording apparatus determines whether an image signal obtained by an endoscope contains a threshold value or more of red color, and recording of the image signal is automatically started when determining that the image signal contains the threshold value or more of red color, and recording of the image signal is automatically stopped when determining that the image signal does not include the threshold value or more of the red color.

WO No. 2021/144951 discloses an image processing apparatus that infers whether an endoscopic image is an image of a predetermined target part by using an arithmetic operation with an artificial intelligence (AI), and automatically records endoscopic images according to an observation mode of endoscopic images.

### SUMMARY

An endoscopy support apparatus according to one aspect of the present disclosure includes a memory and a processor. The processor comprising hardware, wherein the processor is configured to: access a memory that stores: a learned model learned by a learning data set corresponding to a plurality of endoscopic images obtained by imaging an inside of a stomach, wherein the learning data set comprises a name of a part of the stomach, a visual field direction and an axis direction annotated to each one of the plurality of endoscopic images; a name of at least one target part; and a positional relationship of a plurality of the parts of the stomach; input a picked-up image into the learned model; run the learned model to infer a name of a part in the picked-up image, a visual field direction in the picked-up image and an axis direction in the picked-up image; and determine a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

A method of operating an endoscopy support apparatus according to one aspect of the present disclosure includes: accessing a memory that stores: a learned model learned by a learning data set corresponding to a plurality of endoscopic images obtained by imaging an inside of a stomach, wherein the learning data set comprises a name of a part of the stomach, a visual field direction and an axis direction annotated to each one of the plurality of endoscopic images; a name of at least one target part; and a positional relationship of a plurality of the parts of the stomach; inputting a picked-up image into the learned model; running the learned model to infer a name of a part in the picked-up image, a visual field direction in the picked-up image and an axis direction in the picked-up image; and determining a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

A storage medium according to one aspect of the present disclosure is A non-transitory computer-readable storage medium that stores an endoscopy support program causing a computer to execute processing of: accessing a memory that stores: a learned model learned by a learning data set corresponding to a plurality of endoscopic images obtained by photographing an inside of a stomach, wherein the learning data set comprises a name of a part of the stomach, a visual field direction and an axis direction annotated to each one of the plurality of endoscopic images; a name of at least one target part; and a positional relationship of a plurality of the parts of the stomach; inputting a picked-up image into the learned model; running the learned model to infer a name of a part in the picked-up image, a visual field direction in the picked-up image and an axis direction in the picked-up image; and determining a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

### BRIEF DESCRIPTION OF THE DRAWINGS

FIG. 1 is an overall configuration view of an endoscope system including an endoscopy support apparatus in an embodiment.

FIG. 2 is a configuration view of the endoscope system including an endoscopy support apparatus in a first embodiment.

FIG. 3 is a flowchart of a method of operating the endoscopy support apparatus in the first embodiment.

FIG. 4 is a view for describing names of parts of the stomach and a visual field direction of an endoscope.

FIG. 5 is a view for describing the names of the parts of the stomach.

FIG. 6A is a view showing a positional relationship of the parts of the stomach when the visual field direction is a looking-down direction.

FIG. 6B is a view showing a positional relationship of the parts of the stomach when the visual field direction is a looking-up (J-turn) direction.

FIG. 6C is a view showing a positional relationship of the parts of the stomach when the visual field direction is a looking-up (U-turn) direction.

FIG. 7 is an example of a learning data set.

FIG. 8 is an example of a navigation screen.

FIG. 9 is a configuration view of an endoscope system including an endoscopy support apparatus in a second embodiment.

FIG. 10 is a flowchart of a method of operating the endoscopy support apparatus in the second embodiment.

FIG. 11 is a configuration view of an endoscope system including an endoscopy support apparatus in a third embodiment.

FIG. 12 is a flowchart of a method of operating the endoscopy support apparatus in the third embodiment.

FIG. 13 is a configuration view of an endoscope system including an endoscopy support apparatus in a fourth embodiment.

FIG. 14 is a flowchart of a method of operating the endoscopy support apparatus in the fourth embodiment.

FIG. 15 is a configuration view of an endoscope system including an endoscopy support apparatus in a fifth embodiment.

### DETAILED DESCRIPTION OF THE PREFERRED EMBODIMENTS

### First Embodiment

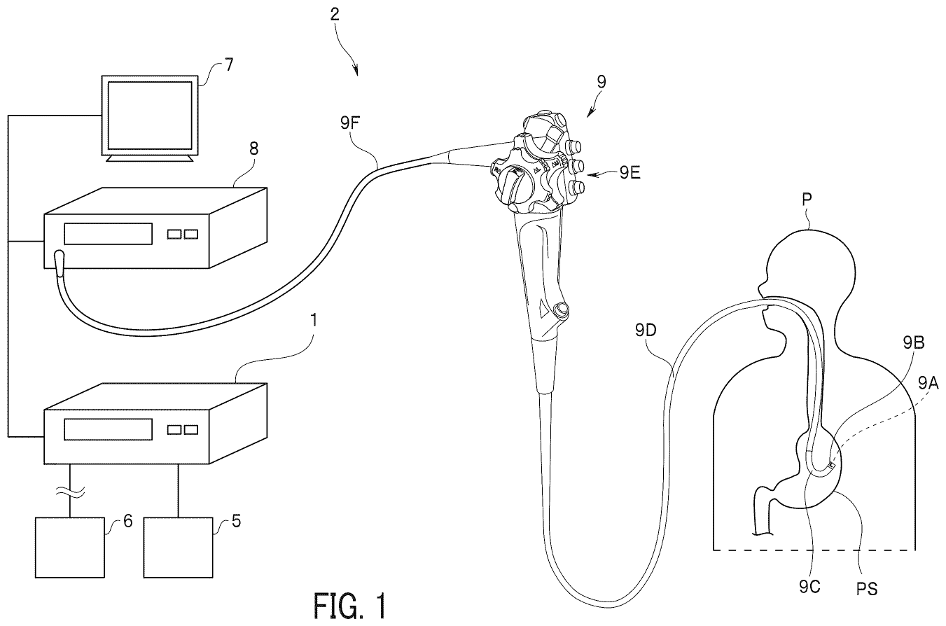

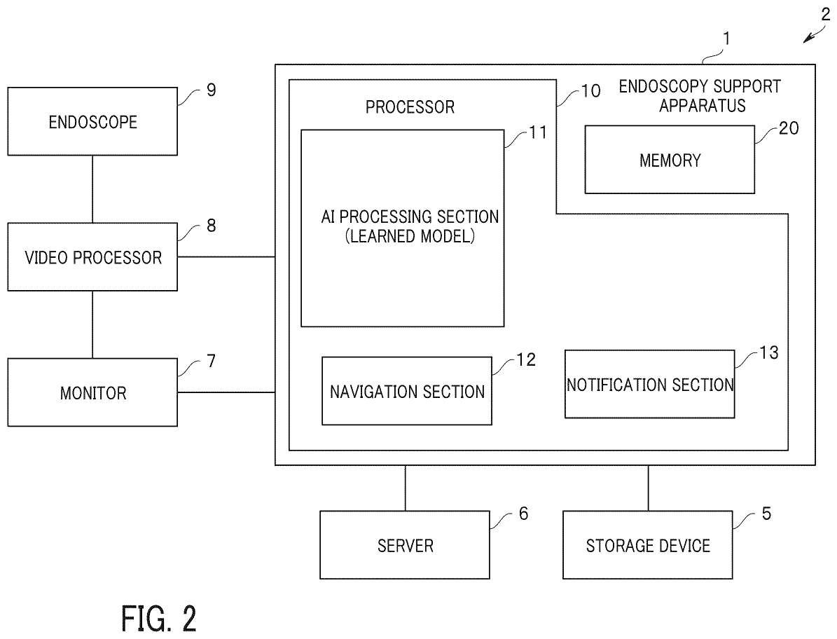

As shown in FIG. 1 and FIG. 2, an endoscopy support apparatus 1 (hereinafter, referred to as “support apparatus 1”) in the present embodiment constitutes an upper gastrointestinal endoscope system 2 (“endoscope system 2”), together with an endoscope 9, a video processor 8, a monitor 7, a storage device 5, and a server 6. The storage device 5 and the server 6 may be shared with other medical apparatuses, and the like, and are not essential components of the support apparatus 1.

In the description below, the drawings based on each embodiment are schematic. The relationship between thicknesses and widths of respective parts, a ratio of a thickness of a certain part to that of another part, a relative angle and the like of the respective parts are different from the actual ones. The respective drawings include parts in which the relationships and ratios among the dimensions are different. In addition, illustration of some constituent elements will be omitted.

The endoscope 9 includes: a distal end portion 9B in which an image pickup unit 9A is disposed; a bending tube 9C configured to be bendable and change a direction of the distal end portion 9B; a flexible tube 9D extended from the bending tube 9C, an operation portion 9E, and a universal cord 9F extended from the operation portion 9E. An operator operates the operation portion 9E, to bend the bending tube 9C, and thereby be capable of changing the direction of the distal end portion 9B inserted into a stomach PS of a subject to be examined P, i.e., the visual field direction of the image pickup unit 9A.

The universal cord 9F is connected to the video processor 8 with a connector. The video processor 8 controls the entire endoscope system 2, performs signal processing on an image pickup signal outputted from the image pickup unit 9A, and outputs a picked-up image. The monitor 7 displays the picked-up image outputted from the video processor 8. The monitor 7 is a display such as a liquid crystal monitor, for example. In the present specification, the “picked-up image” means an “endoscopic image” that is outputted from the endoscope 9 during the examination.

The support apparatus 1 reads a program stored in the storage device 5, which can be a non-transitory storage device (for example, a magnetic disk, an optical disk), or the server 6 connected via a line such as the Internet, to thereby support an endoscopy of the stomach PS of the subject to be examined P.

As shown in FIG. 2, the support apparatus 1 includes a processor 10 and a memory 20. The processor 10 as a CPU includes an AI processing section (AI processing circuit) 11, a navigation section (navigation circuit) 12, and a notification section (notification circuit) 13.

As will be described later, for example, the memory 20 as a RAM stores a learned model, names of target parts, and the like, which are used for the AI processing section 11 to perform inference. The learned model that has been created in advance may be transferred from the storage device 5 and the like to the memory 20.

The AI processing section 11 inputs a picked-up image into the learned model, to thereby infer a name of a part, a visual field direction, and an axis direction, in the picked-up image. Based on the name of the part, the visual field direction, and the axis direction, in the picked-up image, which have been inferred by the AI processing section 11, the navigation section 12 outputs a direction of a target part in the picked-up image. Based on the output data from the navigation section 12, the notification section 13 outputs the direction of the target part to the monitor 7 or the video processor 8, as data that can be displayed on the monitor 7.

All or a part of the functions of the processor 10 may be configured using a logical circuit or an analog circuit. Alternatively, various kinds of processing may be carried out by an electronic circuit such as an FPGA (Field Programmable Gate Array), etc. In addition, the processor 10 may be configured by a plurality of semiconductors. For example, the AI processing section 11 may be a semiconductor dedicated to AI processing.

### <Method of Operating Support Apparatus>

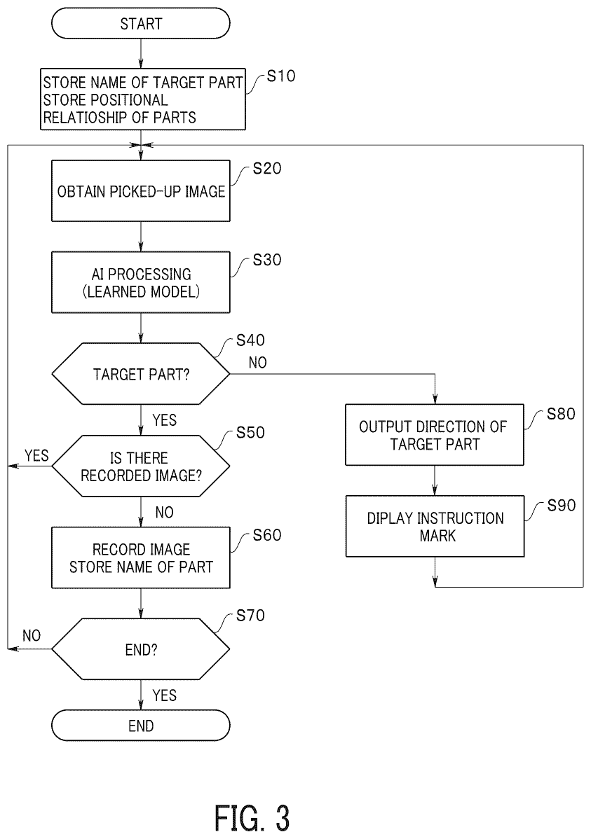

Hereinafter, a method of operating the support apparatus 1 will be described with reference to the flowchart shown in FIG. 3.

### <Step S10> Store Name of Target Part/Store Positional Relationship of Parts

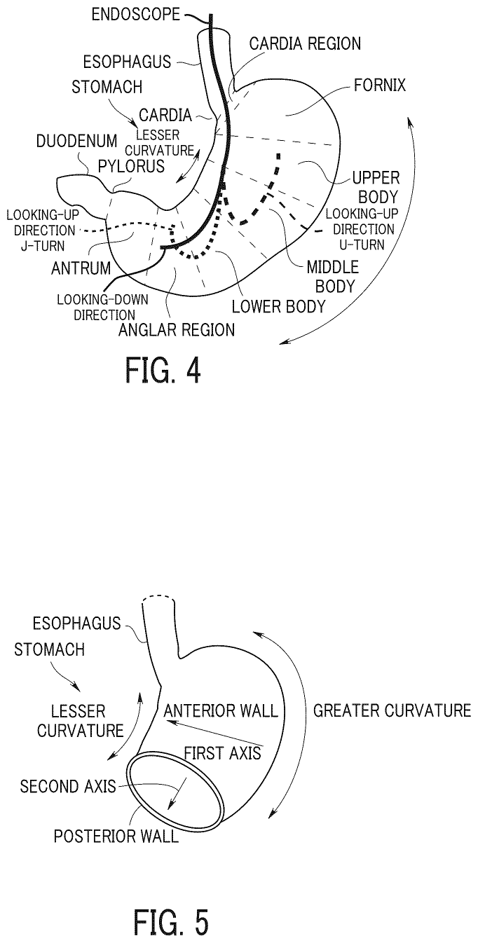

First, FIG. 4 and FIG. 5 show the names of the parts of the stomach PS. The classification of the parts and the names of the parts are not limited to those shown in the drawings. Note that FIG. 4 shows a visual field direction of the endoscope, and FIG. 5 shows an axis direction.

The visual field direction is a “looking-up direction toward the cardia” or a “looking-down direction toward the pylorus”. The “looking-up direction” is further classified into a J-turn in which the distal end portion is turned in the direction of the lesser curvature” and a U-turn in which the distal end portion is turned in the direction of the greater curvature”.

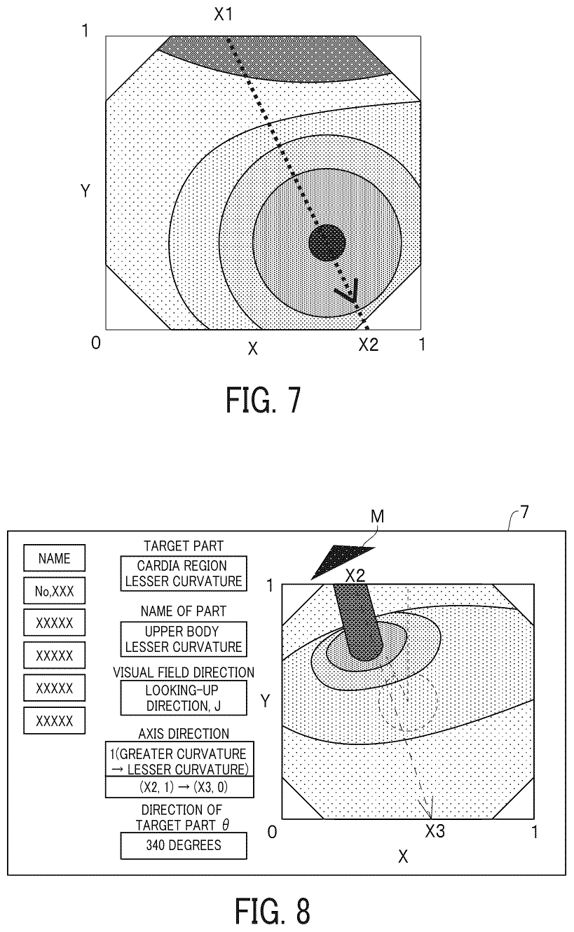

The axis direction is at least either a first axis direction from the greater curvature toward the lesser curvature or a second axis direction from the anterior wall toward the posterior wall. In FIG. 7, coordinates on both ends of the first axis in an XY coordinate system of the picked-up image are used as the axis direction. However, the axis direction is not limited to this. Needless to say, the first axis direction may be a direction from the lesser curvature toward the greater curvature and the second axis direction may be a direction from the posterior wall toward the anterior wall.

The order of observation/photographing in an endoscopy differs depending on the operator. Hereinafter, description will be made by taking the case where the observation is performed in the following order as an example, in which the operator observes a plurality of examination parts anterogradely from the cardia with the observation visual field being in the looking-down direction toward the pylorus, to reach the pylorus, then turns the observation visual field in the looking-up direction toward the cardia, to observe the plurality of examination parts, then returns to the cardia.
1. upper body (greater curvature, anterior wall, posterior wall)
2. middle body (greater curvature, anterior wall, posterior wall)
3. lower body (greater curvature, anterior wall, posterior wall)
4. greater curvature of anglar region (angulus)
5. antrum (greater curvature, anterior wall, posterior wall), pylorus (J-turn operation)
6. lesser curvature of antrum
7. anglar region (lesser curvature, anterior wall, posterior wall)
8. lower body (lesser curvature, anterior wall, posterior wall)
9. middle body (lesser curvature, anterior wall, posterior wall)
10. upper body (lesser curvature, anterior wall, posterior wall)
11. cardia region (anterior wall, posterior wall, lesser curvature) (U-turn operation)
12. cardia region (greater curvature), fornix

The relative positional relationship of the plurality of parts is stored in the memory 20.

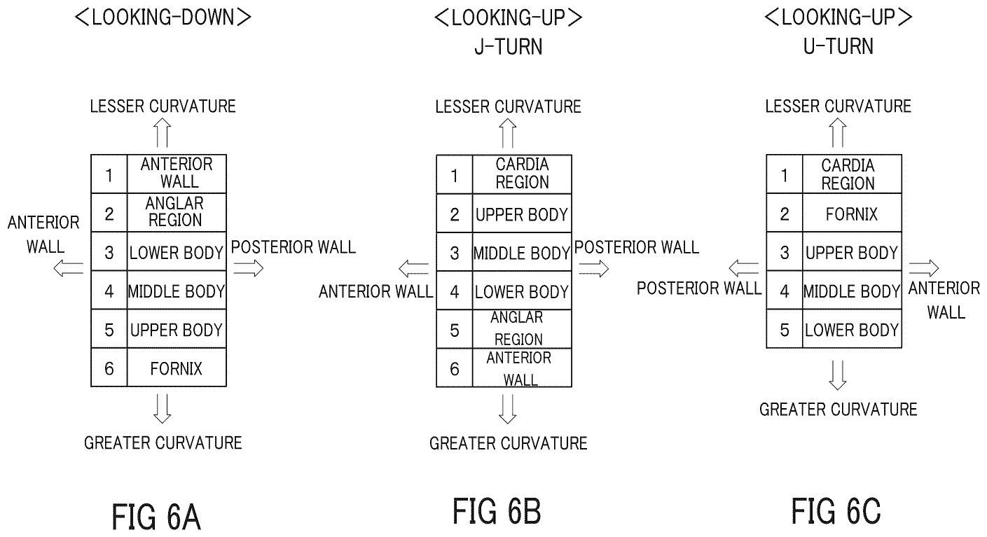

FIGS. 6A to 6C show the relative positional relationship of the plurality of parts in the endoscopic visual field (image). In the respective drawings, the positional relationship is shown, with the direction with the smaller number indicating the upper direction in the drawings, and the direction with the larger number indicating the lower direction in the drawings.

The support apparatus 1 can set all of the plurality of parts, as the target parts to be navigated. However, in the parts that can be observed easily and do not require any navigation, the navigation is complicated for the operator. To address such a problem, the target parts can be set by the operator, for example.

Among the plurality of examination parts, the posterior wall of the anglar region and the lesser curvature of the cardia region are, in particular, difficult to observe, and likely to be a dead angle. In view of the above, hereinafter, description will be made on the case where the posterior wall of the anglar region and the lesser curvature of the cardia region are target parts to be navigated, as an example.

### <Step S20> Obtain Picked-Up Image

The distal end portion 9B of the endoscope 9 is inserted into the stomach PS of the subject to be examined P, and an image of the inside of the stomach PS (hereinafter, referred to as the “picked-up image”) is obtained by the image pickup unit 9A. The picked-up image is a still image automatically clipped from a moving image, or an image photographed by an operation by the operator.

### <Step S30> AI Processing

In the support apparatus 1, the learned model is created in advance. The learned model is learned (for example, deep learning) using a plurality of teaching data (learning data set) in which the name of the part, the visual field direction, and the axis direction are annotated to each of the plurality of endoscopic images (teaching images) obtained by photographing the inside of the stomach. The annotation is performed by an experienced operator.

The annotation of the part is performed, for example, by an annotation of an image classification, a segmentation, a bounding box, or a key point annotation.

By way of example, the name of the part “antrum lesser curvature”, the visual field direction “looking-down direction”, and the axis direction “first axis, (X1, 1)→(X2, 0)” can be annotated to the endoscopic image shown in FIG. 7.

The created learned model is stored in the memory 20. The AI processing section 11 uses the learned model, to infer the name of the part, the visual field direction, and the axis direction for the picked-up image inputted from the video processor 8.

### <Step S40> Target Part?

If the picked-up image for which the AI processing section 11 has performed inference is an image of the target part for navigation processing (YES), the processor 10 performs the processing in the step S50 and subsequent steps. On the contrary, if the picked-up image is not an image of the target part (NO), the processor 10 performs the navigation processing in the step S80 and subsequent steps.

### <Step S50> Is There Recorded Image?

For example, the memory 20 stores the names of the target parts already inferred by the AI processing section 11 in the present examination of the subject to be examined P (see S60). If the name of the part in the picked-up image is already recorded in the memory 20 (YES), the processor 10 repeats the processing in the step S20 and subsequent steps. In other words, a new picked-up image is processed. If no picked-up image is recorded (NO), processing in the step S60 is performed.

### <Step S60> Record Image, Store Name of Part

The picked-up image is stored in the memory 20 or in the storage device 5. In addition, the name of the part in the stored picked-up image is stored in the memory 20.

### <Step S70> End?

Until the observation/photographing/image storing of all of the target parts end (YES), the processing in the step S20 and subsequent steps is repeatedly performed by the processor 10.

When not only the name of the target part but also the name of the examination part is stored in the memory 20, and the picked-up image is inferred as an image of the target part or an image of the examination part in the step S40, the processing in the step S50 and the step S60 may be performed.

### <Step S80> Output Direction of Target Part

Based on the name of the part, the visual field direction, and the axis direction, in the picked-up image, which have been inferred, the navigation section 12 calculates a direction of the target part in the picked-up image.

In other words, the navigation section 12 outputs the direction of the target part based on the data of the positional relationship of the plurality of parts shown in FIGS. 6A to 6C. For example, as described later, the direction of the target part is expressed by an angle θ, with the center point of the picked-up image as a reference.

In the example shown in FIG. 6B, when the visual field direction is (looking-up, J-turn), and the lesser curvature of the upper body is observed, the present position is the lesser curvature of “2: the upper body”. The lesser curvature of “1: cardia region” as the target part is located in the upper direction with respect to the lesser curvature of “2: the upper body”.

### <Step S90> Display Instruction Mark

The notification section 13 outputs the direction of the target part which is outputted from the navigation section 12 to the monitor 7 or the video processor 8. For example, as shown in FIG. 8, an arrow mark M indicating the direction of the target part is displayed with patient information and the like around the picked-up image on the monitor 7.

In FIG. 8, for explanation, the name of the part, the visual field direction, and the axis direction, and also the direction (shown in the drawing) of the target part, in the picked-up image, are displayed on the monitor 7. However, these may not be displayed. In addition, the direction of the target part is indicated with the angle θ in the clockwise direction, with the center of the picked-up image as an origin and the upper direction being an angle of 0 degree. However, the displaying direction is not limited to this. For example, the direction of the target part may be indicated by four directions, i.e., “up, down, right, and left directions”, or eight directions, i.e., diagonal directions (for example, upper right diagonal direction, etc.), in addition to the above-described four directions. In addition, the direction of the target part may be notified of the operator by voice instead of the arrow mark M.

When the operator operates the endoscope 9 with reference to the arrow mark indicating the direction of the target part, to obtain a new picked-up image, the processing in the step S20 and subsequent steps is repeatedly performed.

As described above, the method of operating the endoscopy support apparatus in the embodiment includes: inputting the picked-up image into the learned model learned by the learning data set in which the name of the part, the visual field direction, and the axis direction are annotated to each of the plurality of endoscopic images obtained by photographing the inside of the stomach, to thereby infer the name of the part, the visual field direction, and the axis direction, in the picked-up image; and outputting the direction of the predetermined target part in the picked-up image, based on the positional relationship of the plurality of parts, which is stored in advance, and the name of the part, the visual field direction, and the axis direction, in the picked-up image, which have been inferred.

The endoscopy support program in the embodiment causes a computer to execute: inputting the picked-up image into the learned model learned by the learning data set in which the name of the part, the visual field direction, and the axis direction are annotated to each of the plurality of endoscopic images obtained by photographing the inside of the stomach, to thereby infer the name of the part, the visual field direction, and the axis direction, in the picked-up image; and outputting the direction of the predetermined target part in the picked-up image, based on the positional relationship of the plurality of parts, which is stored in advance, and the name of the part, the visual field direction, and the axis direction, in the picked-up image, which have been inferred.

According to the embodiment of the present disclosure, it is possible to provide the endoscopy support apparatus that enables easy observation of the target part in the stomach, the method of operating the endoscopy support apparatus that enables easy observation of the target part in the stomach, and the endoscopy support program that enables easy observation of the target part in the stomach.

### Second Embodiment

The second embodiment to be described below is similar to the first embodiment and has the same effects as those of the first embodiment. Therefore, the same constituent elements having the same functions as those in the first embodiment are attached with the same reference signs and descriptions thereof will be omitted.

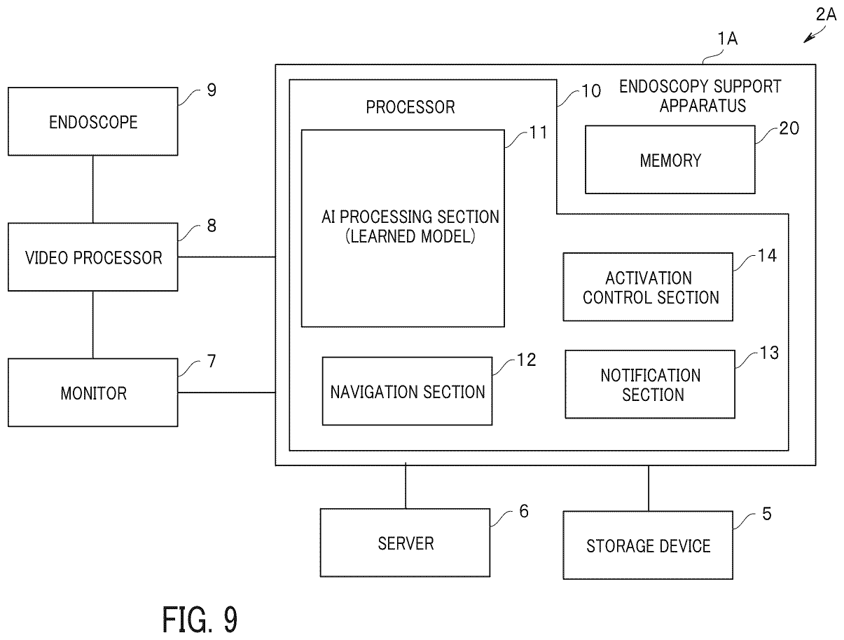

As shown in FIG. 9, a support apparatus 1A in the present embodiment includes an activation control section 14. When the operator observes/photographs a plurality of parts sequentially, if a part in the distance is set as the target part, the navigation by the support apparatus is complicated for the operator.

In the support apparatus 1A, the memory 20 stores names of adjacent parts that are adjacent to the target part. The activation control section 14 controls the AI processing section 11 to infer the direction of the target part in a case where the picked-up image is an image of the adjacent part. In other words, when the operator observes/photographs a plurality of parts sequentially, the navigation processing is performed when approaching close to the target part.

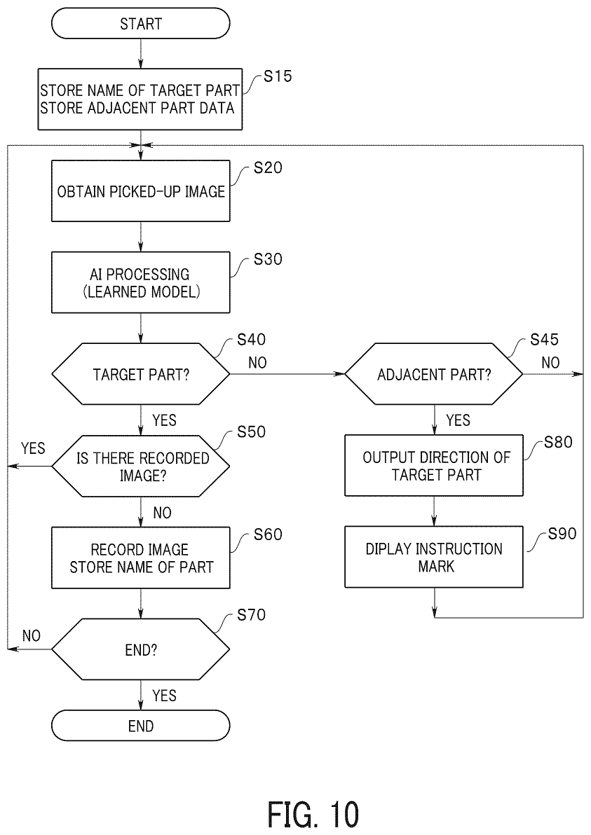

A method of operating the support apparatus 1A will be described with reference to the flowchart in FIG. 10. Since the flowchart in FIG. 10 is the same as the flowchart in FIG. 3, except for the step S15 and the step S45, descriptions thereof will be omitted.

### <Step S15> Store Name of Target Part/Store Adjacent Part Data

When the name of the target part is set, the processor 10 stores data (names of the parts, relative positions, etc.) of the parts adjacent to the target part in the memory 20.

For example, in a case where the target part is the posterior wall of the anglar region, the adjacent parts are the lesser curvature of the anglar region, the lesser curvature of the antrum, and the posterior wall of the antrum. In addition, in a case where the target part is the lesser curvature of the cardia region, the adjacent parts are the anterior wall of the cardia region, the posterior wall of the cardia region, and the lesser curvature of the upper body.

### <Step S45> Adjacent Part?

The activation control section 14 determines whether the part inferred by the AI processing section 11 is the adjacent part. If the part is the adjacent part (YES), the notification section 13 outputs the direction of the target part in the step S80. If the part is not the adjacent part (NO), the processor 10 repeats the processing in the step S20 and subsequent steps.

The support apparatus 1A does not display, on the monitor 7, the navigation information which is a result of inference of the AI processing section 11, until the examination target reaches the adjacent part adjacent to the target part. Therefore, the support apparatus 1A is capable of providing a more comfortable navigation for the operator than the support apparatus 1.

### Third Embodiment

The third embodiment to be described below is similar to the first and second embodiments and has the same effects as those of the first and second embodiments. Therefore, the same constituent elements having the same functions as those in the first and second embodiments are attached with the same reference signs and descriptions thereof will be omitted.

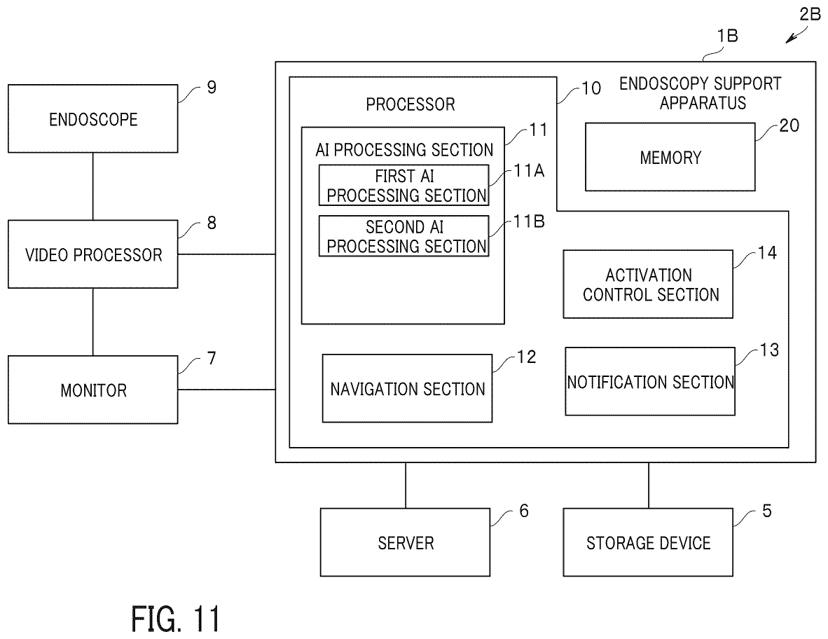

As shown in FIG. 11, in a support apparatus 1B in the present embodiment, the AI processing section 11 includes a first AI processing section 11A and a second AI processing section 11B.

The learned model stored in the memory 20 includes a first learned model learned by a first learning data set in which the name of the part and the visual field direction are annotated, and a second learned model learned by a second learning data set in which the axis direction is annotated. The AI processing section 11 includes a first AI processing section 11A that inputs the picked-up image into the first learned model, to thereby infer the name of the part and the visual field direction, and a second AI processing section 11B that inputs the picked-up image into the second learned model, to thereby infer the axis direction.

Since the first learned model is annotated with the name of the part and the visual field direction, the first learned model can be created in a shorter time and with higher accuracy than the learned model annotated with the name of the part, the visual field direction, and the axis direction. In addition, the first AI processing section 11A is capable of performing inference in a shorter time and with higher accuracy than the AI processing section 11 that performs inference of the name of the part, the visual field direction, and the axis direction. Similarly, the second learned model can be created in a shorter time and with higher accuracy than the learned model annotated with the name of the part, the visual field direction, and the axis direction. In addition, the second AI processing section 11B is capable of performing inference in a shorter time and with higher accuracy than the AI processing section 11 that performs inference of the name of the part, the visual field direction, and the axis direction.

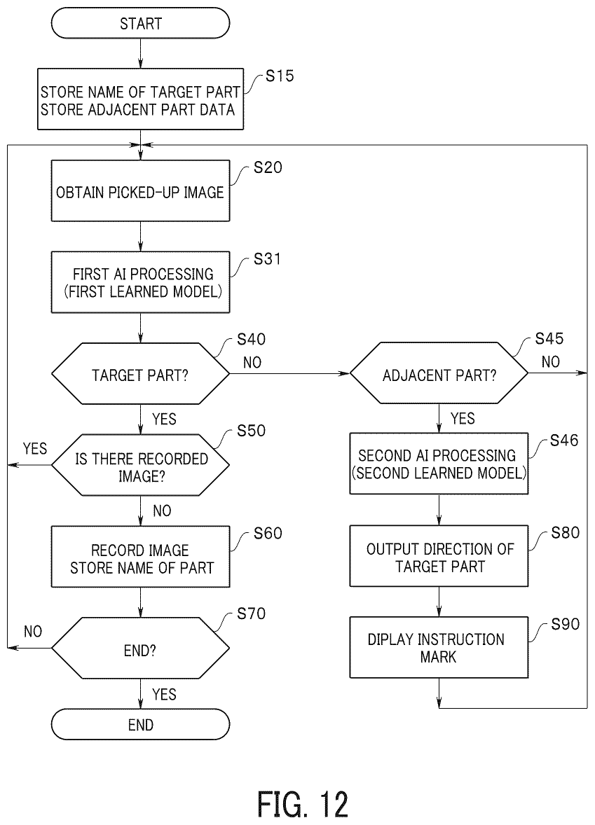

A method of operating the support apparatus 1B will be described with reference to the flowchart in FIG. 12. Since the flowchart in FIG. 12 is the same as the flowchart in FIG. 10 except for the step S31 (S30) and step S46, description thereof will be omitted.

### <Step S31> First AI Processing (First Learned Model)

The first AI processing section 11A uses the first learned model, to infer the name of the part and the visual field direction in the picked-up image inputted from the video processor 8.

### <Step S46> Second AI Processing (Second Learned Model)

The second AI processing section 11B uses the second learned model, to infer the axis direction in the picked-up image.

The support apparatus 1B is capable of performing inference in a shorter time and with higher accuracy than the support apparatus 1A.

### Modified Example of Third Embodiment

Although not shown, in the support apparatus 1B, the second learned model includes a plurality of models in which an axis direction is annotated to each of the names of the parts, and the second AI processing section 11B may input the picked-up image, and the name of the part and the visual field direction, which have been inferred by the first AI processing section 11A, into the second learned model, to thereby infer the axis direction.

The support apparatus in the modified example performs inference using the model of the name of the part inferred by the first AI processing section 11A, among the plurality of models annotated with the axis direction to each of the names of the parts. Therefore, the support apparatus in the modified example is capable of performing inference in a shorter time and with higher accuracy than the support apparatus 1B in the third embodiment that performs inference using the model including all the parts.

### Fourth Embodiment

The fourth embodiment to be described below is similar to the first to third embodiments and has the same effects as those of the first to third embodiments. Therefore, the same constituent elements having the same functions as those in the first to third embodiments are attached with the same reference signs and descriptions thereof will be omitted.

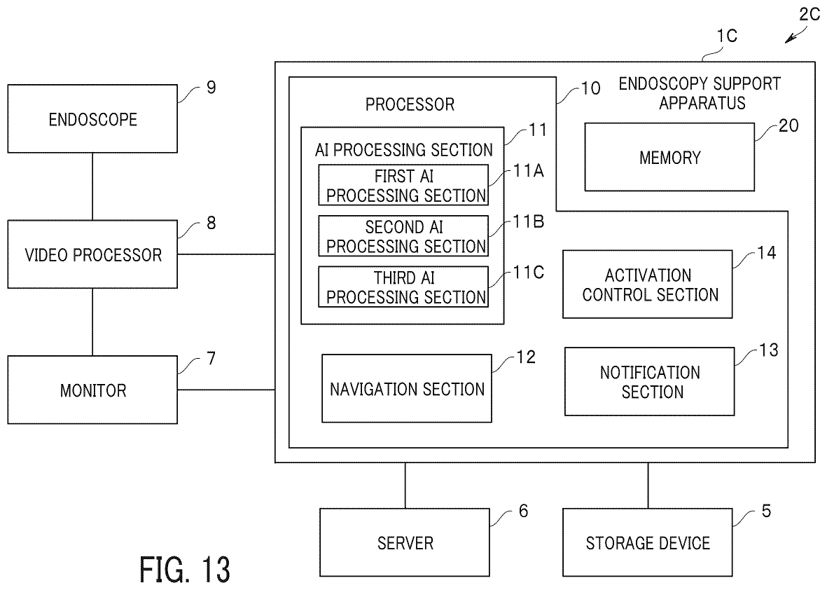

As shown in FIG. 13, in a support apparatus 1C in the present embodiment, an AI processing section 11 includes a first AI processing section 11A, a second AI processing section 11B, and a third AI processing section 11C.

The learned model includes a third learned model learned by a learning data set in which an image quality is annotated to each of endoscopic images. The AI processing section 11 includes a third AI processing section 11C that inputs the picked-up image into the third learned model, to thereby infer the image quality of the picked-up image. When the image quality of the picked-up image satisfies a predetermined quality, the first AI processing section 11A and the second AI processing section 11B perform inference.

For example, the image quality is expressed as a numerical value by a ratio of the area of regions, such as a halation region, an out-of-focus region, a blurred region, and a region of mucus residue adhesion, to the total area of the picked-up image.

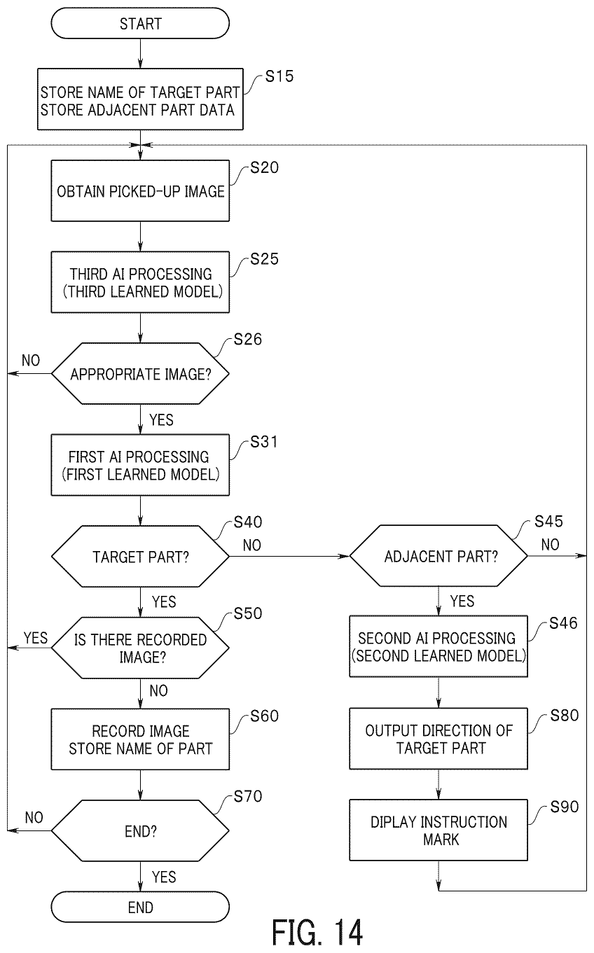

A method of operating the support apparatus 1C will be described with reference to the flowchart in FIG. 14. Since the flowchart in FIG. 14 is the same as the flowchart in FIG. 12 except for the step S25 and the step S26, description thereof will be omitted.

### <Step S25> Third AI Processing (Third Learned Model)

The third AI processing section 11C infers the image quality of the picked-up image using the third learned model.

### <Step S26> Appropriate Image?

If the image quality of the picked-up image satisfies a predetermined quality (YES), the navigation processing starts from the step S31. If the image quality does not satisfy the predetermined quality (NO), the navigation processing is not performed, and the processing in the step S20 and subsequent steps is repeated.

The support apparatus 1C does not perform the navigation processing using an inappropriate picked-up image, to thereby be capable of performing efficient examination. Needless to say, no inappropriate picked-up image is stored in the memory 20.

The processing in the step S25 (third AI processing) may be performed after the processing in the step S50. In other words, the picked-up image may be stored in the memory 20 in the case where the image quality of the picked-up image, which has been inferred by the first AI processing section 11A, satisfies the predetermined quality.

### Fifth Embodiment

The fifth embodiment is similar to the first to fourth embodiments and has the same effects as those of the first to fourth embodiments. Therefore, the same constituent elements having the same functions as those in the first to fourth embodiments are attached with the same reference signs and descriptions thereof will be omitted.

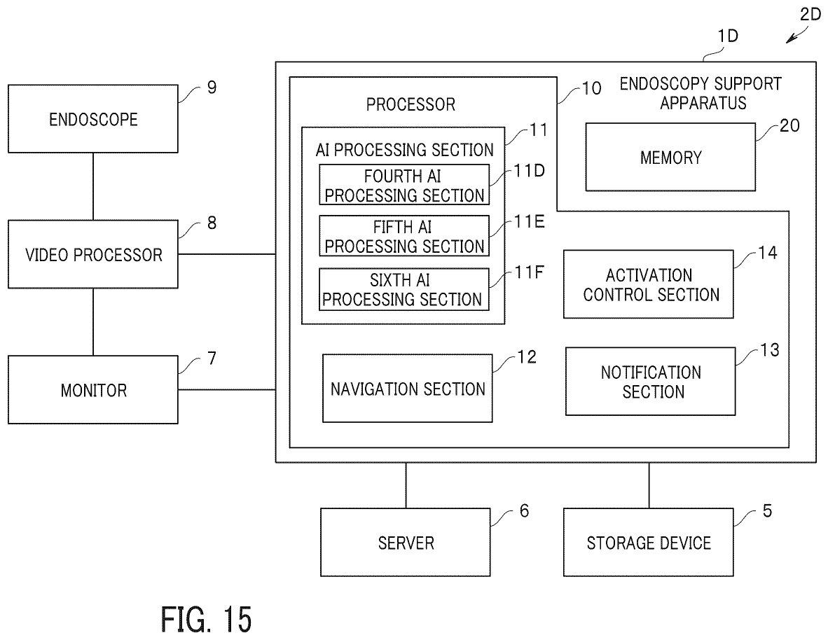

As shown in FIG. 15, in a support apparatus 1D in the present embodiment, an AI processing section 11 includes a fourth AI processing section 11D, a fifth AI processing section 11E, and a sixth AI processing section 11F.

A learned model includes a fourth learned model, a fifth learned model, and a sixth learned model. The fourth learned model is learned by a fourth learning data set in which a name of a part is annotated to each of endoscopic images obtained by photographing an inside of a stomach. The fifth learned model is learned by a fifth learning data set in which a visual field direction is annotated to each of the endoscopic images. The sixth learned model is learned by a sixth learning data set in which an axis direction of at least either a first axis direction from a greater curvature toward a lesser curvature or a second axis direction from an anterior wall toward a posterior wall is annotated to each of the endoscopic images.

The AI processing section 11 infers a direction of a target part by using the fourth learned model, the fifth learned model, and the sixth learned model.

The support apparatus 1D uses a dedicated model for each inference, to perform each AI processing. Therefore, the support apparatus 1D has a smaller circuit, and is capable of performing inference in a shorter time and with higher accuracy than the support apparatus that performs AI processing using the model annotated with a plurality of factors.

It is needless to say that also the support apparatus 1D may eliminate inappropriate images first, and then perform the navigation processing, similarly as the support apparatus 1C.

### <Notes>

The AI processing, etc., may be executed on a server 6, for example. In other words, the learned models are stored in the server 6, and the processor 10 transmits the inputted picked-up image to the server 6, and the AI processing is performed on the server 6. Then, the processor 10 may receive a determination result and an inference result from the server 6. In this case, the server 6 is regarded as a part of the processor 10.

A program for causing the endoscopy support apparatuses 1, and 1A to 1D to execute the above-described operations is entirely or partly stored, in a computer-readable state, on a portable medium such as a flexible disk, a CD-ROM, or the like, or a non-transitory computer-readable storage medium such as a hard disk, or the like. In other words, in the non-transitory computer-readable storage medium (external apparatus), a program for causing a computer to execute the endoscopy support program in the embodiments is stored.

The entirety or a part of the program can be distributed or provided via a communication network. It is possible for a user to implement the support apparatus according to the present disclosure by downloading the program through a communication network to install the program into a computer, or installing the program from a recording medium into the computer.

The respective steps in the flowchart in the disclosure may be performed by changing the execution order, or a plurality of steps may be executed simultaneously as long as they do not deviate from the gist of the disclosure.

The present disclosure is not limited to the above-described embodiments and the like, and various changes and modifications are possible in a range without changing the gist of the present disclosure.

### Example

1. An endoscopy support apparatus comprising,
   a memory and a processor,
   the memory being configured to store:
      a learned model learned by a learning data set in which a name of a part, a visual field direction, and an axis direction are annotated to each of a plurality of endoscopic images obtained by photographing an inside of a stomach; and
      a name of at least one target part and a positional relationship of a plurality of parts,
   the processor comprising:
      an AI processing section configured to input a picked-up image into the learned model, to thereby infer the name of the part, the visual field direction, and the axis direction, in the picked-up image; and
      a navigation section configured to output a direction of the target part in the picked-up image, based on the positional relationship of the plurality of parts, and the name of the part, the visual field direction, and the axis direction, in the picked-up image, which have been inferred by the AI processing section.
2. The endoscopy support apparatus according to example 1, wherein
   the visual field direction is a looking-up direction toward a cardia, or a looking-down direction toward a pylorus.
3. The endoscopy support apparatus according to example 2, wherein
   the axis direction is at least either a first axis direction from a greater curvature toward a lesser curvature, or a second axis direction from an anterior wall toward a posterior wall.
4. The endoscopy support apparatus according to example 1, wherein
   the processor further comprises a notification section configured to output the direction of the target part such that a mark is displayed around the picked-up image displayed on a monitor.
5. The endoscopy support apparatus according to example 1, wherein
   the target part includes at least either a posterior wall of an anglar region or a lesser curvature of a cardia region.
6. The endoscopy support apparatus according to example 1, wherein
   the memory stores the name of the part inferred by the AI processing section, and
   the processor stores the picked-up image in the memory when the name of the target part is not stored in the memory.
7. The endoscopy support apparatus according to example 6, wherein
   the memory stores a name of an adjacent part that is adjacent to the target part, as the positional relationship of the plurality of parts, and
   the processor includes an activation control section configured to control the AI processing section to infer the direction of the target part only in a case where the picked-up image is an image of the adjacent part.
8. The endoscopy support apparatus according to example 3, wherein
   the learned model includes a first learned model and a second learned model, the first learned model being learned by a first learning data set in which the name of the part and the visual field direction are annotated, the second learned model being learned by a second learning data set in which the axis direction is annotated, and
   the AI processing section includes a first AI processing section and a second AI processing section, the first AI processing section being configured to input the picked-up image into the first learned mode to thereby infer the name of the part and the visual field direction, the second AI processing section being configured to input the picked-up image into the second learned model to thereby infer the axis direction.
9. The endoscopy support apparatus according to example 8, wherein
   the second learned model includes a plurality of models annotated with the axis direction for each name of the part, and
   the second AI processing section is configured to input the picked-up image, and the name of the part and the visual field direction that are inferred by the first AI processing section into the second learned model, to thereby infer the axis direction.
10. The endoscopy support apparatus according to example 8, wherein
   the learned model includes a third learned model learned by a learning data set in which an image quality is annotated to each of the endoscopic images,
   the AI processing section includes a third AI processing section configured to input the picked-up image into the third learned model, to thereby infer the image quality of the picked-up image, and
   the picked-up image is stored in a case where the image quality of the picked-up image satisfies a predetermined quality.
11. The endoscopy support apparatus according to example 1, wherein
   the learned model includes:
      a fourth learned model learned by a fourth learning data set in which the name of the part is annotated to each of the endoscopic images obtained by photographing the inside of the stomach;
      a fifth learned model learned by a fifth learning data set in which the visual field direction is annotated to each of the endoscopic images; and
      a sixth learned model learned by a sixth learning data set in which the axis direction, which is at least either a first axis direction from a greater curvature toward a lesser curvature or a second axis direction from an anterior wall toward a posterior wall, is annotated to each of the endoscopic images, and
      the AI processing section is configured to use the fourth learned model, the fifth learned model, and the sixth learned model, to infer the direction of the target part.
12. A method of operating an endoscopy support apparatus comprising:
   inputting a picked-up image into a learned model learned by a learning data set in which a name of a part, a visual field direction, and an axis direction are annotated to each of a plurality of endoscopic images obtained by photographing an inside of a stomach, to thereby infer the name of the part, the visual field direction, and the axis direction, in the picked-up image; and
   outputting a direction of a predetermined target part in the picked-up image, based on a positional relationship of a plurality of parts, which is stored in advance, and the name of the part, the visual field direction, and the axis direction, in the picked-up image, which have been inferred.
13. An endoscopy support program causing a computer to execute processing of:
   inputting a picked-up image into a learned model learned by a learning data set in which a name of a part, a visual field direction, and an axis direction are annotated to each of a plurality of endoscopic images obtained by photographing an inside of a stomach, to thereby infer the name of the part, the visual field direction, and the axis direction, in the picked-up image; and
   outputting a direction of a predetermined target part in the picked-up image, based on a positional relationship of a plurality of parts, which is stored in advance, and the name of the part, the visual field direction, and the axis direction, in the picked-up image, which have been inferred.

What is claimed is:

1. An endoscopy support apparatus comprising:
a processor comprising hardware, wherein the processor is configured to:
access a memory that stores:
a learned model learned by a learning data set corresponding to a plurality of endoscopic images obtained by imaging an inside of a stomach, wherein the learning data set comprises a name of a part of the stomach, a visual field direction and an axis direction annotated to each one of the plurality of endoscopic images;
a name of at least one target part; and
a positional relationship of a plurality of the parts of the stomach;
input a picked-up image into the learned model;
run the learned model to infer a name of a part in the picked-up image, a visual field direction in the picked-up image and an axis direction in the picked-up image; and
determine a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

2. The endoscopy support apparatus according to claim 1, wherein:
the visual field direction of the learning data set is a looking-up direction toward a cardia, or a looking-down direction toward a pylorus.

3. The endoscopy support apparatus according to claim 2, wherein:
the axis direction of the learning data set is at least one of a first axis direction from a greater curvature toward a lesser curvature and a second axis direction from an anterior wall toward a posterior wall.

4. The endoscopy support apparatus according to claim 1, wherein:
the processor is further configured to control a monitor to display the picked-up image and a mark around the picked-up image identifying the direction of the at least one target part.

5. The endoscopy support apparatus according to claim 1, wherein:
the at least one target part includes a posterior wall of an anglar region or a lesser curvature of a cardia region.

6. The endoscopy support apparatus according to claim 1, wherein
the processor is further configured to:
determine whether the inferred name of the part in the picked-up image is the name of the at least one target part;
in response to determining that the inferred name of the part in the picked-up image is not the name of the at least one target part, determine the direction of the at least one target part in the picked-up image; and
in response to determining that the inferred name of the part in the picked-up image is the name of the at least one target part:
determine whether a previously acquired image of the at least one target part is stored in the memory; and
in response to determining that the image of the at least one target part is not stored in the memory, store the picked-up image as an image of the at least one target part.

7. The endoscopy support apparatus according to claim 6, wherein:
the memory is configured to store a name of an adjacent part that is adjacent to the at least one target part, as the positional relationship of the plurality of parts; and
the processor is further configured to:
determine whether the inferred name of the part in the picked-up image is the name of the at least one target part; and
in response to determining that the inferred name of the part in the picked-up image is not the name of the at least one target part:
determine whether the inferred name of the part in the picked-up image is the name of the adjacent part; and
in response to determining that the inferred name of the part in the picked-up image is the name of the adjacent part, infer the direction of the target part based on positional relationship of the adjacent part and the at least one target part.

8. The endoscopy support apparatus according to claim 3, wherein:
the learned model includes a first learned model and a second learned model, the first learned model being learned by a first learning data set including names of parts and visual field directions annotated in the plurality of endoscopic images, and the second learned model being learned by a second learning data set including axis directions annotated in the plurality of endoscopic images; and
the processor is configured to run the first learned model to infer the name of the part in the picked-up image and the visual field direction in the picked-up image, and run the second learned model to infer the axis direction in the picked-up image.

9. The endoscopy support apparatus according to claim 8, wherein:
the second learned model includes a plurality of models annotated with the axis direction for each name of the part; and
the processor is configured to:
input the picked-up image, and the inferred name of the part and the inferred visual field direction into the second learned model; and
run the second learned model to infer the axis direction in the picked-up image.

10. The endoscopy support apparatus according to claim 8, wherein:
the learned model includes a third learned model learned by a learning data set including an image quality annotated to each one of the endoscopic images; and
the processor is configured to:
input the picked-up image into the third learned model;
run the third learned model to infer the image quality of the picked-up image; and
store the picked-up image in a case where an image quality of the picked-up image satisfies a predetermined quality.

11. The endoscopy support apparatus according to claim 1, wherein:
the learned model includes:
a fourth learned model learned by a fourth learning data set including the name of the part annotated to each one of the endoscopic images obtained by photographing the inside of the stomach;
a fifth learned model learned by a fifth learning data set including the visual field direction annotated to each one of the endoscopic images; and
a sixth learned model learned by a sixth learning data set including the axis direction, which is at least either a first axis direction from a greater curvature toward a lesser curvature or a second axis direction from an anterior wall toward a posterior wall, annotated to each one of the endoscopic images; and
the processor is further configured to run the fourth learned model, the fifth learned model, and the sixth learned model, to infer the direction of the at least one target part.

12. A method of operating an endoscopy support apparatus comprising:
accessing a memory that stores:
a learned model learned by a learning data set corresponding to a plurality of endoscopic images obtained by imaging an inside of a stomach, wherein the learning data set comprises a name of a part of the stomach, a visual field direction and an axis direction annotated to each one of the plurality of endoscopic images;
a name of at least one target part; and
a positional relationship of a plurality of the parts of the stomach;
inputting a picked-up image into the learned model;
running the learned model to infer a name of a part in the picked-up image, a visual field direction in the picked-up image and an axis direction in the picked-up image; and
determining a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

13. A non-transitory computer-readable storage medium that stores an endoscopy support program causing a computer to execute processing of:
accessing a memory that stores:
a learned model learned by a learning data set corresponding to a plurality of endoscopic images obtained by photographing an inside of a stomach, wherein the learning data set comprises a name of a part of the stomach, a visual field direction and an axis direction annotated to each one of the plurality of endoscopic images;
a name of at least one target part; and
a positional relationship of a plurality of the parts of the stomach;
inputting a picked-up image into the learned model;
running the learned model to infer a name of a part in the picked-up image, a visual field direction in the picked-up image and an axis direction in the picked-up image; and
determining a direction of the at least one target part in the picked-up image, based on the positional relationship of the plurality of parts of the stomach, the inferred name of the part in the picked-up image, the inferred visual field direction in the picked-up image, and the inferred axis direction in the picked-up image.

14. The method according to claim 12, wherein:
the visual field direction of the learning data set is a looking-up direction toward a cardia, or a looking-down direction toward a pylorus.

15. The method according to claim 14, wherein:
the axis direction of the learning data set is at least one of a first axis direction from a greater curvature toward a lesser curvature and a second axis direction from an anterior wall toward a posterior wall.

16. The method according to claim 12, further comprising
controlling a monitor to display the picked-up image and a mark around the picked-up image identifying the direction of the at least one target part.

17. The method according to claim 12, wherein:
the at least one target part includes a posterior wall of an anglar region or a lesser curvature of a cardia region.

18. The method according to claim 12, further comprising:
determining whether the inferred name of the part in the picked-up image is the name of the at least one target part;
in response to determining that the inferred name of the part in the picked-up image is not the name of the at least one target part, determining the direction of the at least one target part in the picked-up image; and
in response to determining that the inferred name of the part in the picked-up image is the name of the at least one target part:
determining whether a previously acquired image of the at least one target part is stored in the memory; and
in response to determining that the image of the at least one target part is not stored in the memory, storing the picked-up image as an image of the at least one target part.

19. The non-transitory computer-readable storage medium according to claim 13, wherein:
the visual field direction of the learning data set is a looking-up direction toward a cardia, or a looking-down direction toward a pylorus.

20. The non-transitory computer-readable storage medium according to claim 13, wherein:
the axis direction of the learning data set is at least one of a first axis direction from a greater curvature toward a lesser curvature and a second axis direction from an anterior wall toward a posterior wall.
